(19) United States
(12) Patent Application Publication
(10) Pub. No.: US 2025/0288392 A1
(43) Pub. Date: Sep. 18, 2025

(54) TOOTH MOVEMENTS

(71) Applicants: Mohammedreza Sefidroodi, Aarhus (DK); Stratos Vassis, Aarhus (DK); Beatrice Noeldeke, Aarhus (DK)

(72) Inventors: Mohammedreza Sefidroodi, Aarhus (DK); Stratos Vassis, Aarhus (DK); Beatrice Noeldeke, Aarhus (DK)

(21) Appl. No.: 18/607,613

(22) Filed: Mar. 18, 2024

(51) Int. Cl. A61C 7/00; G06T 7/00; G16H 10/60

(52) U.S. Cl. CPC A61C 7/002; G06T 7/0014; G16H 10/60; G06T 2207/20081; G06T 2207/30036

(57) ABSTRACT

A method of training a machine learning model to determine tooth movements for a process of tooth alignment of a subject. The method comprises receiving, in a computer memory, for a plurality of subjects: planned tooth movements for a tooth alignment process for aligning one or more teeth of the respective subject; and actual tooth movements for said one or more teeth of the respective subject after a tooth alignment process has been performed. The method further comprises training one or more machine learning models at least in part using the planned tooth movements. The one or more machine learning models are arranged to extract one or more predicted deviations based at least in part on the planned tooth movements. The or each predicted deviations represent differences between the planned tooth movements and expected tooth movements if the planned tooth movements were used in a tooth alignment process.

### FIELD OF THE INVENTION

There are provided methods of training a machine learning model to improve tooth movements for a process of tooth alignment of a subject. There are also provided methods of determining improved tooth movements for a process of tooth alignment of a subject, using the trained machine learning model. There are also provided systems for performing said methods.

### BACKGROUND

Tooth alignment processes for straightening (or aligning) teeth are popular. The present disclosure relates in particular to a type of tooth alignment process called aligner treatment, commonly referred to as clear aligner therapy. One example of a provider of this type of tooth alignment process is Invisalign®.

An aligner treatment typically involves wearing a series of clear, removable plastic trays, called aligners or aligner trays. Each aligner is formed to the shape of the subject's teeth and/or mouth and arranged to apply a gentle and consistent pressure to the teeth to gradually move the teeth into or towards desired positions. Typically, aligner trays are clear and so are discrete. Furthermore, aligner trays are usually removable. This makes eating, drinking and, importantly tooth cleaning (e.g. brushing/flossing) straightforward. This contrasts with traditional metal braces which are intrusive in appearance and permanently fixed in place. Thus, aligner treatment has become more popular in recent times.

Aligner treatment requires a customized treatment plan. The customized treatment plan typically involves taking impressions, photographs, and/or digital scans of the teeth of a subject to create a 3D model. Using specialized aligner software, the movements of teeth will be planned in order to move the subject's teeth from their initial or starting position to a desired final position. Based on the planned movements, a series of custom-made aligners will be fabricated specifically for each subject. Often, the series of aligners comprises between about 10 to 40 plastic trays for each jaw. Each aligner is designed to gradually shift teeth slightly closer to the desired position. Aligners must be worn for about 20 to 22 hours a day, removing them only to eat, drink (except water), brush, and floss. It is important to follow the prescribed wear schedule to achieve optimal results.

A problem with current aligner methods is that the subject's teeth often do not move as planned. In other words, the actual tooth movements of the subject often do not match the planned tooth movements such that aligner trays of the original plan do not correctly move the subject's teeth into the desired positions. Thus, there is a need for periodic check-up appointments throughout the treatment process to monitor progress and ensure that teeth are moving as planned (also known as tracking). If teeth are not moving as planned, there may be a need for the plan to be refined with another set of aligners. This might include fabricating further or different aligner trays to either over- or under-compensate for an actual tooth movement not going as planned. This often necessitates multiple supplementary sets of aligners in additional treatment rounds which in turn requires new scans to be made.

One of the reasons for the deviations between planned and actual tooth movements is that current aligner software for orthodontic tooth movements frequently proves to be imprecise, with a study finding mean accuracy of tooth movement in current plans to be only 50%. This is because current software is imprecise in predicting the complex and often non-linear movements of teeth in response to an applied pressure. The prediction of tooth movements is particularly complicated because individual teeth have many different degrees of freedom of motion (e.g. translation in various directions and/or rotation about multiple different axes of rotation). Inclination and rotational movements are the most unpredictable movements. Furthermore, the motion of each individual tooth also depends on the surrounding teeth. So, when predicting tooth movements, individual teeth cannot generally be modelled as independent features, instead the (complex) behavior of the mouth as a whole must be taken into account (as a complex system). Thus, while current aligner software may plan for a flawless alignment of a subject's teeth, the actual outcome will typically deviate from this, creating the necessity of overcorrecting or potentially reducing some tooth movements in the aligner software as described above in subsequent rounds/after subsequent appointments.

There is a need to improve the precision of planned tooth movements in tooth alignment procedures (in particular, aligner treatments) to allow for the fabrication of better aligner trays that more accurately move teeth into desired positions.

### BRIEF SUMMARY OF THE INVENTION

In view of the above, an objective of the present invention is to overcome or at least mitigate drawbacks of current tooth alignment procedures.

In general terms, there is provided a method of training a machine learning model (e.g. a neural network) to determine tooth movements (or improved tooth movements) for a process of tooth alignment of a subject. There is provided a method and system that uses the trained machine learning model to determine improved planned tooth movements as part of a tooth alignment process. Said method may comprise, for example, fabricating aligner trays for a process of tooth alignment based on the improved planned tooth movements.

In more detail, the inventors have recognised that machine learning models such as neural networks are well-suited to capturing the complex patterns and relations inherent in the complex and difficult-to-predict systems that are teeth and tooth movements. In contrast to traditional methods, neural networks excel at learning datasets where the relationship between input and output is complex and nonlinear, making them well-suited for predicting tooth movements. The inventors have therefore recognised that machine learning models can be used to improve planned tooth movement.

In the present disclosure, the inventors have proposed using planned tooth movements output by current (non-machine learning based) software solutions to train a machine learning model. The machine learning model is arranged to extract predicted deviations representing differences (e.g. errors or deviations) between the planned tooth movements output by the current software solution and expected tooth movements that would occur if the originally planned tooth movements were used in an alignment process—e.g. if a series of aligner trays were fabricated and used in an alignment process. The differences or deviations can then be used to improve the planned tooth movements. For example, the machine learning model might predict that a planned inclination of a first tooth of a subject is an overestimate. In other words, the machine learning model might predict that the actual movement of the first tooth is too much. Based on this prediction, the planned tooth improvements can be improved by reducing the planned inclination of the first tooth. For example, if the initially planned inclination of the first tooth is predicted to overestimate the movement needed by 10 degrees, then this movement may be reduced by 10 degrees in the improved tooth movements. In this way, the inventors have found that the machine learning model of the present disclosure can improve tooth movements for a tooth alignment procedure. The advantage of this is that, when the improved tooth movements are used in an alignment process, there is a much higher chance of achieving the desired alignment the first time. Methods using the improved tooth movements may have a much lower chance of needing subsequent appointments and plan adjustments throughout a treatment plan. At the very least, the number of appointments may be reduced relative to current solutions. This can improve and speed up the alignment process. The reduction in appointments also reduces time and resources needed from the dentist/orthodontist. Furthermore, there is a reduction in the number of aligner trays that need to be manufactured.

In some examples, the training method is a supervised learning model. In such examples, the machine learning model may be trained using a training data set comprising, for a plurality of subjects: a) the planned tooth movements output by a current software solution, and b) the actual tooth movements for the subject when a tooth alignment process (e.g. aligner treatment process) is performed on the subject using the planned tooth movements (without improvement). The difference between a) and b) may be determined and may be referred to as the deviation or error. These deviations may be used in the training. For example, to train the machine learning model to accurately predict the deviation for a given input planned tooth movements.

Alternatively, or additionally, the inventors have recognised that another source of inaccuracy in current solutions is that one or more teeth are used as reference points for the models used to plan teeth movements. The inventors have identified that teeth are not a good reference point because the position of individual teeth during a tooth alignment process is not stable. For example, this might be as a result of moving during the alignment process either as a reaction to a force given to another tooth or calculated planned movements programmed into the aligner. The inventors have recognised that the movements can be improved by using stable non-dental orthodontic reference points. This could include using anatomical landmarks and/or temporary anchorage points, for example. Such reference points may be stable. For example, the position of the reference points may remain substantially unchanged throughout the course of an alignment process.

The inventors have found that these stable reference points may have particular advantage in the preparing of the training data for training the machine learning model, as described above. In particular, the training method may be based at least in part on actual tooth movements. The actual tooth movements may be determined by comparing data from first and second scans of the tooth or teeth of a subject, wherein the first scan is a scan taken before the tooth alignment process using the planned tooth movements and the second scan is a scan taken after said tooth alignment process. The comparison may comprise determining how the teeth have moved between the first and second scans. To do this accurately, it may be important to have a stable reference frame. The inventors have recognised that the non-dental orthodontic reference points are stable reference points that may be used to provide this stable reference frame and so improve the accuracy of the model.

Conventionally, the scan or scans used in an alignment process such as an aligner treatment involves scanning the teeth only. It is unusual, in the context of aligner treatment, to include scan data of other features of the subject (e.g. anatomical landmarks and/or temporary anchorage points). However, the inventors have recognised that there is value in building and using a training dataset comprising such features (and so scanning additional features of the subject) for improved accuracy.

In a first aspect there is provided a method of training a machine learning model to determine tooth movements for a process of tooth alignment of a subject. In some embodiments, the process of tooth alignment is an aligner treatment process such as clear aligner therapy. The method comprises receiving, in a computer memory, for a plurality of subjects:
a) planned tooth movements for a tooth alignment process for aligning one or more teeth of the respective subject;
b) first scan data obtained in a first scan of the respective subject before the tooth alignment process; and
c) second scan data obtained in a second scan of said one or more teeth of the respective subject after the tooth alignment process has been performed.

The first scan data and the second scan data comprise scan data for said one or more teeth of the respective subject. The first scan data and second scan data also comprise scan data for one or more non-dental orthodontic reference points of the respective subject.

The method comprises determining, for one or more of the subjects (optionally, for each subject) actual tooth movements of the respective one or more teeth using the first and second scan data and the non-dental orthodontic reference points.

The method comprises determining, for one or more of the subjects (optionally, for each subject) one or more deviations between the planned tooth movements and the actual tooth movements.

The method comprises training one or more machine learning models. The training at least in part involves inputting planned tooth movements of the one or more subjects. The one or more machine learning models are arranged to extract one or more predicted deviations based at least in part on the planned tooth movements. The or each predicted deviation represents one or more predicted differences between the planned tooth movements and expected tooth movements if the planned tooth movements were used in a tooth alignment process.

In some embodiments, the deviations between the planned tooth movements and the actual tooth movements of the or each subject represent ground truth or target data for the particular planned tooth movements/subject. Thus, in such embodiments, training the model may comprise reducing a difference between the deviations predicted by the machine learning model and the actual deviations (of the ground truth or target data).

As used herein, “planned tooth movements” are tooth movements that have been previously determined or calculated for use as part of a tooth alignment process. For example, the planned tooth movements may have been calculated or determined by scanning a subject's teeth and, optionally, further portions of the subject's mouth and/or face and/or cranium including the non-dental orthodontic reference points. Positions of the subject's teeth may be determined based on the scan data (e.g. from a model formed from data of the scan). The planned tooth movements may be determined by inferring tooth movements that are required to align the subject's teeth in accordance with a desired alignment. In some embodiments, the tooth movements needed to achieve this are calculated manually. In some embodiments, the tooth movements needed to achieve a desired alignment are calculated using software such as a CAD package. In any case, as above, the inventors have found that it is very difficult using current methods to determine or calculate planned tooth movements that accurately result in the desired alignment if the planned tooth movements are used in an actual alignment process.

As above, in some embodiments, the planned tooth movements have been obtained using a computer-aided design CAD package for modelling virtual tooth movement. Such CAD packages may include: ClinCheck Pro® (of Invisalign®), Approver™ (of Spark™ aligners, Ormco Inc) Ortho Analyzer™ (of 3Shape™ inc) and other aligner softwares from companies such as SureSmile® (of Sirona) Angel aligner™ (of Angelaligner™), ClearCorrect™ (of Straumann™), Reveal® Clear Aligners (of Henry Schein® Orthodontics™), Clarity™ (of 3M™), Clear Aligners (of Argen®), Refine® Complete Aligner System (of TP orthodontics®).

As used herein, “actual tooth movements” are the real tooth movements that occurred when a process of tooth alignment is carried out based on the planned tooth movements. As described previously, the unpredictability and complexity of predicting tooth movements means that there is typically a discrepancy or deviation or error between planned tooth movements and actual, real, tooth movements. This means that currently, after a tooth alignment process has been carried, the subject's teeth generally are not in the desired or planned alignment. It is these deviations that the methods of the present disclosure are arranged to quantify/predict and to compensate for.

As used herein, a tooth movement (e.g. a planned tooth movement and/or an actual tooth movement) comprises one or more values representing a change in at least one of: in inclination, angulation, rotation, or translation of the respective tooth from an initial position to a final position. Translational movements may comprise buccolingual translation, intrusion or exclusion. For planned tooth movements, the initial position may refer to a position as determined in an initial scan as part of a tooth alignment process and the final position may refer to a desired (theoretical) position of the tooth. For actual tooth movements, the initial position may refer to a position as determined using the first scan data and the final position may refer to a position as determined using the second scan data. Teeth have many degrees of freedom and so multiple different tooth movement values (describing different degrees of freedom) may be needed to describe the movement (e.g. translational and/or rotational movements) of the tooth from the initial position to the final position.

As used herein, “rotation” refers to rotation of a tooth about a rotation axis or tooth axis. The rotation or tooth axis extends from a midpoint of the mesio-distal line of the tooth to an apex point of the tooth. A rotation tooth movement is a value representing a change in angle of a tooth about the tooth axis from the initial position to the final position of the tooth. A positive value indicates increasing rotation meaning the labial landmark of the tooth is moved/tilt in a mesial direction

As used herein, “inclination” refers to a rotation of a tooth about mesio-distal axis. The mesio-distal axis extends from the mesial arch point to the distal arch point of the tooth. An inclination tooth movement is a value representing a change in angle of a tooth about the mesio-distal axis from the initial position to the final position of the tooth. A positive value indicates increasing inclination meaning that the incisal landmark of the tooth is moved/tilted in a buccal direction and the apex landmark of the tooth is moved/tilted in the opposite direction.

As used herein, “angulation” refers to a rotation of a tooth about a vestibular axis. The visitable axis extend perpendicularly to the above described tooth axis and mesio-distal axis. An angulation tooth movement is a value representing a change in angle of a tooth about the vestibular axis from the initial position to the final position of the tooth. A positive value indicates increasing angulation meaning that an incisal landmark of a tooth is moved/tilted in a mesial direction and an apex landmark of the tooth is moved/tilted in the opposite direction.

As used herein, “buccolingual translation” refers to a translation of a tooth along the above-described vestibular axis. A buccolingual translational tooth movement is a value representing a change in position of a tooth along the vestibular axis. A positive value indicates movement towards the buccal (cheek) region.

As used herein, “intrusion” and “extrusion” refers to an occlusal translation of a tooth along the above-described mesio-distal axis. An occlusal translation is a value representing a vertical change in the mesio-distal axis. A positive value represents extrusion, indicating a movement away from the gingiva, while a negative value represents intrusion indicating a movement towards the gingiva.

The different degrees of freedom of motion may be referred to as different “types” of the tooth movement (e.g. planned or actual). For example, inclination may be a first type of tooth movement. Angulation may be a second type of tooth movement. Rotation may be a third type of tooth movement. Buccolingual translation may be a fourth type of tooth movement. Intrusion and/or extrusion may be a fifth type of tooth movement.

In some embodiments, the method comprises training a machine learning model (e.g. neural network) with regards to a specific type of tooth movement. In some embodiments, the method may comprise training a plurality of machine learning models (e.g. neural networks), each being trained with regards to a specific/unique type of tooth movement. For example, the method may comprise training a first machine learning model with regards to a first type of movement (such as angulation) and training a second machine learning model with regards to a second type of movement (such as buccolingual translation). In some embodiments, the method may comprise training as many machine learning models as there are types of movements to be predicted. Thus, the training method of the first aspect may be repeated to train a plurality of different machine learning models.

In more detail, in some embodiments, the method comprises training a first machine learning model to improve a first type of tooth movement for a process of tooth alignment of a subject; and training a second machine learning model to improve a second type of tooth movements for the process of tooth alignment of a subject

The method of training the first machine learning model may be as described above. In this case, however, the planned tooth movements may be a first type of planned tooth movements (e.g. relating to planned changes in angulation). Similarly, the actual tooth movements that are determined may also be of the first type (e.g. relating to actual changes in angulation). Similarly, the determined deviations may be deviations in the first type of tooth movements (e.g. deviations in angulation). Similarly, the predicted deviations (that the first machine learning model is arranged to extract) may be deviations in the first type of tooth movements (e.g. predicted deviations in angulation if the planned tooth movements were used in a tooth alignment process).

The method of training the second machine learning model may also be as described above. In this case, however, the planned tooth movements may be a second type of planned tooth movements (e.g. relating to planned changes in buccolingual translation). Similarly, the actual tooth movements that are determined may also be of the second type (e.g. relating to actual changes in buccolingual translation). Similarly, the determined deviations may be deviations in the second type of tooth movements (e.g. deviations in buccolingual translation). Similarly, the predicted deviations (that the second machine learning model is arranged to extract) may be deviations in the second type of tooth movements (e.g. predicted deviations in buccolingual translation if the planned tooth movements were used in a tooth alignment process).

Training the first machine learning model may comprise receiving (planned tooth movements and first and second) data for a first plurality of subjects. Training the second machine learning model may comprise receiving (planned tooth movements and first and second) data for a second plurality of subjects. The first plurality of subjects may be partially or completely the same as the second plurality of subjects. The first plurality of subjects may be entirely different to the second plurality of subjects.

As used herein, “scan data”, such as the first and second scan data, refers to data that has been obtained in one or more scans performed on the respective subject. In some embodiments, scan data may comprise intraoral scans or scans of impressions of the teeth of the subject. Said scans optionally also include the surrounding portions of the teeth, e.g. soft tissue and the palate. Alternatively or additionally, scan data may comprise data obtained in a computed tomography (CT) scan. Alternatively or additionally, scan data can comprise data obtained in a magnetic resonance imaging (MRI) scan. If the data comprises data from multiple scan sources (e.g. CT and intraoral) then said scans may be synchronised with one another. As the first and second scan data comprises scan data for one or more teeth of the subject and for one or more non-dental orthodontic reference points, the one or more scans comprise scanning both said one or more teeth of the subject and said reference points. Thus, if the reference point is a palatal reference point, for example, the scanning may comprise scanning said one or more teeth and the portion of the palate of the subject comprising the reference point.

In some embodiments, the first scan data and/or the second scan data comprises data associated with the lower jaw of the respective subject. In some embodiments, the first scan data and/or the second scan data comprises data associated with the upper jaw of the respective subject. In some embodiments, the first scan data and/or the second scan data comprises data associated with the upper and lower jaw of the respective subject.

As used herein, the “non-dental orthodontic reference points” are stable reference points used to enable accurate comparison of one set of tooth data to another set of tooth data (e.g. comparison of the one or more teeth of the first and second scans). This improves the accuracy of the method, as described above. As used herein, “stable means” that the reference point does not substantially move throughout a tooth alignment process. In other words, the position of the reference point is constant in both the first scan data and the second scan data. The position being constant may mean that reference point is fixed in relation to other stable features of the subject such as the palatal rugae, hard tissue anatomical landmarks exemplified by the mandibular canal and/or other structures in the cranium. As used herein “orthodontic reference points” includes natural anatomical landmarks as well as artificial temporary anchorage points. As used herein, “non-dental” means that the reference point is not a tooth or teeth of the subject. In other words, in the present disclosure, teeth are excluded from use as orthodontic reference points. As above, the inventors have found that teeth do not make good reference points because the position of teeth moves during an alignment process and so teeth do not provide stable reference points.

In some embodiments, the one or more non-dental orthodontic reference points comprise at least one of: an anatomical landmark of the respective subject and a temporary anchorage point received by the respective subject.

The one or more non-dental orthodontic reference points may comprise one or more anatomical landmarks comprising at least one of: a feature or portion of the palate such as soft or hard tissue, a feature or portion of the median suture, a feature or portion of the incisive foramen, one or more anterior contours of the chin, one or more trabecular structures of the mandibular canal and symphysis, one or more inner cortical structures at the inferior border of the symphysis, and one or more lower contours of a molar germ.

A temporary anchorage point (TAP), also known as a temporary anchorage device (TAD) or mini-implant, may be a small, biocompatible screw-like device that is temporarily placed into the jaw or other area of the subject's mouth. Such temporary anchorage points or devices may advantageously provide a stable reference point and may be particularly advantageous in the lower jaw where there may be fewer anatomical landmarks suitable for providing a stable reference point than the upper jaw.

In some embodiments, determining the actual tooth movements comprises forming a first model of the respective one or more teeth based on the first data; and forming a second model of the respective one or more teeth based on the second data. This step may be computer-implemented. In other words, the first and second models may be computer or software models such as CAD models. In such embodiments, the first and second scan data may be received at a processor. The processor may be arranged to convert the first and second scan data in the first and second models. In some embodiments however, a manual model may be formed.

The first and second models may comprise models of the one or more teeth. Because the first model is based on the first scan data, the one or more teeth in first model may be representative of the respective teeth of the subject prior to a tooth alignment process being performed. Similarly, because the second model is based on the second scan data, the one or more teeth in the second model may be representative of the respective teeth of the subject after a tooth alignment process has been performed using the respective planned tooth movements.

In some embodiments, the first and second models further comprise the one or more non-dental orthodontic reference points (of the scan data). Thus, the first and second models may comprise a model of the one or more anatomical landmarks and/or one or more temporary anchorage points. The non-dental orthodontic reference points are stable reference points and so do not substantially move during a tooth alignment process. Thus, the reference points can be used as fixed alignment points the are common to both the first and second models. In some embodiments, determining the actual tooth movements comprises superimposing or aligning the non-dental orthodontic reference points of the first model and the non-dental orthodontic reference points of the second model. In some embodiments, determining the actual tooth movements comprises comparing at least one of a position and orientation of a tooth or teeth in first and second models.

In some embodiments, the planned tooth movements and the actual tooth movements comprise movements for a plurality of teeth, optionally at least 10 teeth, optionally at least 20 teeth, optionally all teeth of the respective subject. This may include teeth from either or both of the top and bottom jaws of the respective subject. In some embodiments therefore, the first scan data (and the second scan data) may comprise scan data for a plurality of teeth, optionally at least 10 teeth, optionally at least 20 teeth, optionally all teeth of the respective subject.

In embodiments comprising a plurality of teeth, the machine learning model may be trained to extract the predictions for all teeth/all deviations at once. In other words, the machine learning model may be arranged to treat the plurality of teeth as a complex system, rather than treating individual teeth as individual simple systems. The inventors have recognized that this ability to process a plurality of tooth movements simultaneously is one of the advantages of training a machine learning model to improve predictions/planned tooth movements rather than a more numeric or traditional software approach.

In more detail, tooth movements are influenced by various factors, including neighboring teeth. By considering a plurality of teeth—or the entire mouth—as a holistic entity, the trained machine learning model (e.g. neural network) can capture the interdependencies and interactions among teeth within the dental arch. This comprehensive approach enables the model to leverage contextual information and global patterns present in the entire mouth, which may not be apparent when analyzing teeth in isolation. Moreover, training the machine learning model (e.g. neural network) to process a plurality of teeth—or the entire mouth—simultaneously allows it to learn complex spatial relationships and coordination between different teeth. For example, the movement of one tooth may affect the positioning and alignment of adjacent teeth, necessitating a global understanding to accurately predict the desired outcomes. In addition, increasing the number of teeth considered simultaneously increases the data the model is trained on.

In some embodiments, the method comprises performing the first scan for one or more of the plurality of subjects, optionally all of the plurality of subjects. The method may comprise receiving the data from the first scan in the computer memory. In some embodiments, the method comprises performing the second scan for one or more of the plurality of subjects. The method may comprise receiving the data from the second scan in the computer memory. The first and/second scan may comprise performing at least one of: an intraoral scan, taking an impression of a subjects jaw and scanning the impression, a CT scan, and an MRI scan.

In some embodiments, the first and/or second scans have been performed previously for at least some, optionally all of the plurality of subjects. In such embodiments, data from the first scan(s) and the second scan(s) may be stored in a database. Receiving the first and second scan data may comprise receiving said data from the database, for example.

As above, in some embodiments, the or each machine learning model is a neural network such as a deep neural network. The inventors have recognized that neural networks may be advantageous because neural networks are able to capture complex patterns inherent in the data that might be challenging for numerical methods and/or humans to discern. In contrast to traditional (e.g. numerical) methods, neural networks excel at learning datasets where the relationship between input and outputs is complex and nonlinear, making them well-suited for predicting tooth movements. By feeding the network with movement data, it may autonomously learn to identify subtle patterns and correlations. Deep neural networks, in particular, are adept at hierarchical feature learning, where lower layers learn basic features (e.g., edges, textures) and higher layers learn more abstract and complex features. It is possible to adapt and refine predictions of neural networks over time when new data becomes available, ensuring that planning remains up-to-date and reflective of the latest data. Neural networks are efficient to scale effectively for handling large datasets with a high number of samples and features. They further offer flexibility in architecture design as the number of layers or number of neurons in each layer can be customized. This flexibility allows neural networks to be tailored to specific tasks and datasets.

In some embodiments, the or each machine learning model is a Decision Tree, Random Forest, Gradient Boosting, Support Vector Machine, Regression Model (for example, K-Nearest Neighbors Regression, Ridge Regression, Lasso Regression, ElasticNet Regression), Gaussian Processes, Kernel Density Estimation, Nearest Centroid Classifier, Generalized Additive Models, Recurrent Neural Networks or an Ensemble Model.

In a second aspect, there is provided a method of training a machine learning model to improve tooth movements for a process of tooth alignment of a subject. The method comprises receiving, in a computer memory, for a plurality of subjects:
a) planned tooth movements for a tooth alignment process for aligning one or more teeth of the respective subject; and
b) actual tooth movements for said one or more teeth of the respective subject after a tooth alignment process has been performed.

The method further comprises training one or more machine learning models at least in part using the planned tooth movements, the one or more machine learning models being arranged to extract one or more predicted deviations based at least in part on the planned tooth movements.

The or each predicted deviations represent differences between the planned tooth movements and expected tooth movements if the planned tooth movements were used in a tooth alignment process.

In some embodiments, the method comprises determining deviations between the planned and actual tooth movements for each subject. The training of the model may comprise optimizing the model to reduce a difference between the determined deviations (between planned and actual movements for a subject) and predicted deviations output/extracted by the model.

Features and advantages described in relation to the first aspect may also be applicable to the second aspect.

In a third aspect, there is provided a system for training a machine learning model to improve tooth movements for a process of tooth alignment of a subject. The system comprises one or more processors and a memory comprising instructions which, when executed by the one or more processors, cause the system to carry out the step of receiving, for a plurality of subjects:
a) one or more planned tooth movements for a tooth alignment process for aligning one or more teeth of the respective subject;
b) first scan data obtained in a first scan of the respective subject before the tooth alignment process; and
c) second scan data obtained in a second scan of said one or more teeth of the respective subject after a tooth alignment process has been performed.

Both the first and second scan data comprises scan data for said one or more teeth of the respective subject and scan data for one or more non-dental orthodontic reference points of the respective subject.

The steps (performed by the system) further comprise determining, for one or more of the subjects, actual tooth movements of the respective one or more teeth using the first and second scan data and the non-dental orthodontic reference points.

The steps (performed by the system) further comprise determining deviations between the planned tooth movements and the actual tooth movements.

The steps (performed by the system) further comprise training one or more machine learning models at least in part using planned tooth movements of one or more subjects, the one or more machine learning models being arranged to extract one or more predicted deviations based at least in part on the planned tooth movements.

The or each predicted deviation represents one or more predicted differences between the planned tooth movements and expected tooth movements if the planned tooth movements were used in a tooth alignment process.

In a fourth aspect, there is provided a system for determining (improved) tooth movements for a process of tooth alignment of a subject. The system comprises one or more processors and a memory comprising instructions which, when executed by the one or more processors, cause the system to carry out the following steps.

The steps comprise receiving one or more planned tooth movements for a tooth alignment process for aligning one or more teeth of the respective subject one or more planned tooth movements of one or more teeth of the subject.

The steps comprise extracting, using a machine-learned model trained in accordance with the method as describe in the first or second aspect, one or more predicted deviations based at least in part on the planned tooth movements.

The steps comprise determining improved planned tooth movements. The or each predicted deviation represents one or more differences between the planned tooth movements and expected tooth movements if the planned tooth movements were used in a tooth alignment process. The improved planned tooth movements are based at least in part on the planned tooth movements and the predicted deviations.

In some embodiments, determining improved planned tooth movements comprises combining the planned tooth movements and the predicted deviations. This may comprise adding or subtracting the predicted deviations to the respective planned tooth movements to output the improved planned tooth movements.

In some embodiments, the system further comprises a communication device. In such embodiments, the system may be arranged to carry out the step of communicating, using the communication device, data associated with the improved tooth movements to a fabrication device for manufacturing an aligner tray.

In some embodiments, the system is arranged to perform the step of creating or updating a CAD model for manufacturing an aligner tray using the improved planned tooth movements.

In some embodiments, the system is arranged to perform the step of fabricating, using a fabrication device, one or more aligner trays in accordance with the improved tooth movements. This may comprise 3D printing the one or more aligner trays, for example.

In a fifth aspect, there is provided method of determining improved tooth movements for a process of tooth alignment of a subject. The method comprises receiving, at a processor, one or more planned tooth movements of one or more teeth of the subject. The method comprises extracting, using a machine learning model trained in accordance with the method as described in the first and second aspect, one or more predicted deviations based at least in part on the planned tooth movements. The method comprises determining, using a processor, improved planned tooth movements.

The or each predicted deviation represents one or more differences between the planned tooth movements and expected tooth movements if the initial planned tooth movements were used in a tooth alignment process. The improved planned tooth movements are based at least in part on the planned tooth movements and the predicted deviations.

Although different embodiments may be disclosed separately in the detailed description which follows, any feature of any embodiment may be combined with any other feature or combination of features of any embodiment. That is, all possible combinations and permutations of features disclosed in the present disclosure are envisaged.

### BRIEF DESCRIPTION OF THE DRAWINGS

Specific embodiments are described by way of example only with reference to the following figures:

FIG. 1 is a schematic diagram of an example of a method of training a machine learning model according to the present disclosure;

FIG. 2 is a flow diagram illustrating the method of training the machine learning algorithm;

FIG. 3 is a flow diagram 300 showing sub-sets of the method of FIG. 2, in particular the steps of determining discrepancies between planned and actual tooth movements;

FIG. 4A illustrates a front view of a tooth and represents various movements of the tooth;

FIG. 4B illustrates a side view of the tooth of FIG. 4A;

FIG. 5 shows a first model of the top jaw of a subject including soft tissue structures;

FIG. 6 shows a second model of the top jaw of a subject including hard tissue structures;

FIG. 7 shows a third model of the top jaw of a subject including soft and hard tissue structures;

FIG. 8 shows a fourth model of the top jaw of a subject including temporary anchorage points;

FIG. 9 shows a flow chart of a method of determining improved tooth movements using the machine learning algorithm trained as per the method of FIG. 2;

FIGS. 10A to 10D represent some stages of the process of determining improved tooth movements; and

FIG. 11 illustrates a tooth movement unit for performing the methods of FIG. 2 and/or 9.

### DETAILED DESCRIPTION

According to embodiments of the present invention as disclosed herein, the above-mentioned disadvantages of solutions according to prior art are eliminated or at least mitigated.

Current teeth alignment processes such as aligner treatment typically involve devising a customised treatment plan based on a 3D model of the subject's teeth (which might be built up using impressions, photographs and/or digital scans of the subject). Tooth movements are then planned, typically using software which determines planned tooth movements that aim to achieve desired positions and orientations of the mouth, generally to achieve an aesthetically pleasing or desired alignment of the teeth. A series of aligner trays are then fabricated based on the planned tooth movements which are intended to be used in turn, with each aligner tray being arranged to apply pressure and/or torque to one or more of the subject's teeth with the aim of moving the teeth towards the desired alignment.

A problem is that, currently, after planned treatment, a subject's teeth often are not in the desired alignment. There is a discrepancy between the planned teeth movements that the aligner trays were designed to achieve and the actual teeth movements that occurred during the treatment process. The reason for this is that teeth movements are complicated and difficult to predict. The movement of one tooth can have effects on the movement of adjacent teeth and so on. Current approaches (e.g. current software) typically falls short in terms of modelling this complicated behaviour. Thus, currently, there is a need for periodic appointments and scans throughout treatment to check planned movement progress. It is often necessary to fabricate further or different aligner trays to over or under compensate for actual tooth movements not going as planned.

The machine learning model of the present disclosure can address these problems. In particular, the inventors have recognised that a machine learning model can be trained to predict the discrepancy between the planned teeth movements output by current methods (e.g. current software) and the actual teeth movements that result from an aligner treatment process using those planned teeth movements. This predicted discrepancy can then be used to output improved planned tooth movements.

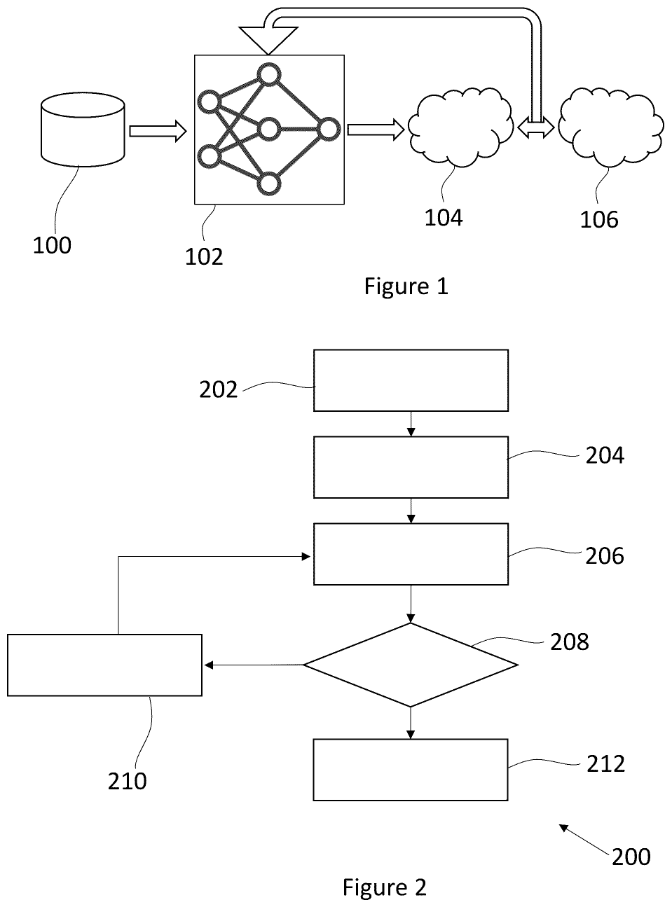

FIG. 1 is a schematic diagram of an example of a method of training a machine learning model according to the present invention to improve tooth movements for a process of tooth alignment of a subject. In this example, the tooth alignment process is an aligner treatment process. In this example, the machine learning model is a deep neural network.

The diagram of FIG. 1 comprises memory 100 and a machine learning algorithm 102. The memory is arranged to store training data for training the machine learning algorithm 102. The training data comprises training data from a plurality of a training subjects. The training data is derived primarily from subjects who have gone through the process of having a teeth aligner treatment. In this example, for each subject, the training data comprises planned tooth movements for a tooth alignment process for aligning the teeth of each subject. In this example, the planned tooth movements have been output by third-party software such as ClinCheck Pro® of Invisalign®. Importantly, the planned tooth movements of the training data have not been improved by a machine learning model according to the present disclosure.

Each subject has been through a tooth alignment process in which aligner trays are fabricated based on the planned tooth movements. In this example, the training data also comprises, for each subject, deviations between the planned tooth movements and the actual tooth movements that occurred after the respective subject has undergone an aligner treatment plan based on the planned tooth movements (output by the third-party software, in this example). As will be explained in more detail below, the discrepancies may be determined based on scan data from each subject before and after the tooth alignment process has been carried out to determine actual tooth movements, and then comparing the actual tooth movements to the planned tooth movements. In this example, the training data represents the situation after a tooth alignment process has been carried in which the planned tooth movements do not change throughout the course of the treatment. In particular, in this example, the planned tooth movements and/or aligner trays were not changed periodically throughout the process. In other words, the training data represents a “first round” of conventional treatment. As described previously, often conventional treatment requires further rounds with further scans and further aligner sets to compensate for inaccuracies in tooth movements after the first round. These further rounds do not form part of the training data (in this example).

The machine learning algorithm 102 is arranged to receive as input planned teeth movements for a first subject from the training data and to output a predicted deviation 104. The predicted deviation 104 is the machine learning algorithm's 102 estimation as to the deviations that would exist if the planned teeth movements were used to fabricate aligner trays and if an alignment process using those trays was followed. The training method then comprises comparing the predicted deviation 104 to an actual deviation 106 for that first subject from the training data which, in this example, is stored in memory 102. The actual deviation 106 could be described as a target or ground truth. The training method comprises comparing the predicted deviation 104 to the actual deviation 106. In examples, this comprises determining a difference or loss function between the two. If the difference is greater than a predetermined threshold, then the method comprises optimizing the machine learning algorithm (e.g. by optimising the weights of the algorithm) and a new predicted deviation is output. This process is repeated either for a set number of iterations or until the difference/loss is reduced to below the predetermined threshold.

The training method is repeated for each of a plurality of the subjects of the training data.

FIG. 2 is a flow diagram 200 illustrating the method of training of the machine learning algorithm 102.

Step 202 comprises receiving, at memory 100, for each of plurality of training subjects, planned tooth movements for an aligner treatment process for aligning the teeth. In examples, there are 50 or more, optionally 100 or more training subjects. As the skilled reader will appreciate, the greater the number of training subjects, the larger the amount of training data that is available and the better the trained model will be at predicting teeth deviations. However, this must be balanced with the burden (e.g. computationally and from an admin perspective) of processing a very large number of training subjects. In this example, the received planned tooth movements are output by ClinCheck Pro® of Invisalign®. However, it should be understood that the planned tooth movements could be output from software from any provider of aligner treatment/clear aligners.

Step 204 comprises receiving, at memory 100, for each of the plurality of training subjects, discrepancies or deviations between the planned tooth movements and the actual tooth movements that occurred after a tooth alignment process in which the planned tooth movements received in step 202. In this example, the tooth movement alignment process is a process performed by Invisalign® using the planned tooth movements of step 202. The discrepancies received at step 204 of the method is used as the target or ground truth during the training.

Step 206 comprises using the machine learning algorithm 102 to extract predicted deviations based on the planned tooth movements of a first subject of the plurality of subjects.

Step 208 comprises comparing the predicted deviations of step 206 with the respective (actual) deviations for that subject received at step 204 of the method. In this example, the comparison comprises using a loss function to quantify a difference between the predicted deviations of step 206 and actual deviations received at step 204. If the difference is greater than a threshold, then the method moves to step 210 which is to optimize the machine learning algorithm 102 (e.g. by updating weights of the algorithm 102). The method then returns to step 206 where the method is repeated. If the difference is less than (or equal to) the threshold, then the method moves to step 212 which is to stop training (at least based on the first subject).

The training method (or, at least, steps 206 onwards) may be repeated for each of the plurality of subjects. In some examples, training based on multiple subjects may performed simultaneously.

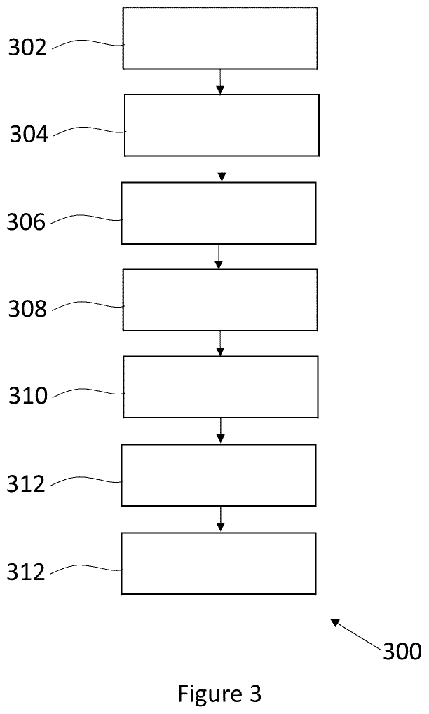

In some examples, step 204 comprises further steps in order to determine the discrepancies received as part of step 204. This is outlined in FIG. 3 which is a flow diagram 300 illustrating the steps of determining the discrepancies for a first subject of the plurality of subject.

Step 302 of the method of FIG. 3 comprises receiving first scan data obtained in a first scan of a first subject. The first scan is a scan that was taken before a tooth alignment process has been performed.

Step 304 of the method comprises receiving second scan data obtained in a second scan of the first subject. The second scan is a scan that was taken after a tooth alignment process. The tooth alignment process is a tooth alignment process using the planned tooth movements for the respective subject (as received in step 202).

In examples, the first and/or second scans each comprise two intraoral scans or impressions of the upper and lower teeth of the subject. The scans also include the surrounding soft tissue of the palate. In examples in which impressions are taken, the impression(s) is scanned after being made.

Alternatively, or additionally, the first and/or second scans each comprise two cone beam computed tomography scans of the hard tissue structures of the cranium.

Alternatively, or additionally, the first and/or second scans comprise two MRI scans.

Step 306 of the method comprises forming a first model of the teeth of the first subject using the data obtained in the first scan (from step 302). As the first model is based on the first scan data, the first model is a model of the subject's teeth before the teeth alignment process. Step 308 of the method comprises forming a second model of the teeth of the first subject using the data obtained in the second scan (from step 304). As the second model is based on the second scan data, the second model is a model of the subject's teeth after the teeth alignment process.

In examples, step 306 and/or step 308 comprise uploading the data obtained in the first and/or second scan into computer software such as such as OnyxCeph™ (Image Instruments, Chemnitz, Germany), where a segmentation procedure is performed.

In examples in which the scan data comprises data from a CT scan, the first and second can then be generated into a 3D format exemplified by the STL file format. In examples comprising scan data from two different scan types, such as from a CT scan and from an intraoral scan, the STL file format of the CT scan the data from the intraoral scan can be synchronized by superimposition on tooth structures in the oral cavity.

Step 310 of the method comprises aligning or superimposing the first and second models with one another. This comprises using non-dental orthodontic reference points present in both the first and second models and basing the alignment or superimposition on those reference points (by aligning or superimposing the reference points).

In more detail, the first and second scan data of step 302 and 304 comprises scan data related to one or more stable non-dental orthodontic reference points. The reference points are temporary or permanent, anatomical or artificial features of the subject that are scanned in the first and second scans. The stable non-dental orthodontic reference points do not substantially move during a tooth alignment process and so are reliable reference points that can be used in the superposition or alignment.

Because the first and second scan data of steps 302 and 304 comprises scan data related to the non-dental orthodontic reference points, the first and second models of steps 306 and 308 also can comprise the non-dental orthodontic reference points. In some examples, the non-dental orthodontic reference points are picked up in a first type of scan that is different to a second type of scan for scanning the teeth. In such cases, it is important to make sure that the different types of scan are synchronized with one another, as described above.

In one specific example, using OnyxCeph™, the superposition or alignment can be achieved by: uploading the scan data into OnyxCeph™, then open the first model in the software's aligner module, where the second scan is uploaded in the “load” under “Register step” section of the module. The actual superimposition can be done by placing point coordinates or marking an area of the models comprising the reference points. The software then utilizes a best fit algorithm based on the marked area in both digital models when “Align” is pressed.

Step 312 of the method comprises determining actual teeth movements based on the superimposed first and second models. The tooth movements such as the amount of inclination, rotation, translation, vestibular, intrusive and extrusive movements are recorded compared to the tooth's initial position. Thus, values for these movements are relative, indicating the relative change of the tooth from the initial position.

In one specific example, using OnyxCeph™, determining actual teeth movements comprises superimposing the first and second models as described above, and then pressing “comparison” and “registration”. This enables a tooth movement table to be generated.

Step 314 of the method comprises determining the differences between the planned teeth movements and the actual teeth movements which are then received at memory 100 as part of step 204.

The process of FIG. 3 may be performed for each subject of a plurality of subjects of the training data in order to determine the differences between the planned teeth movements and the actual teeth movements.

Teeth have a number of different degrees of freedom of motion (e.g. translation and rotation in various directions and about various axes). Each of these forms a different type of movement. Each teeth may have a value for a (planned or actual) tooth movement for each of type of movement. In the present example, the types of movement we are considering include: inclination, angulation, rotation, m/d translation (along a mesial/distal axis), b/l translation (along a buccal/lingual axis), and occlusal translation.

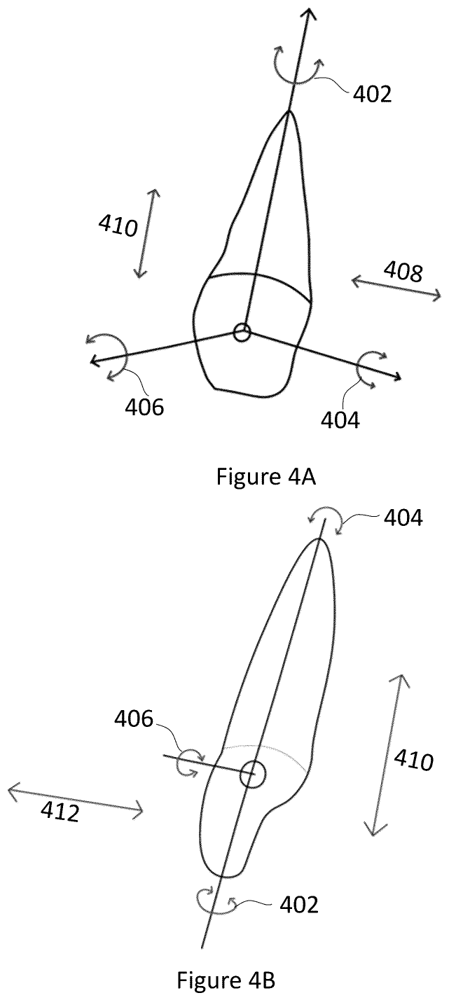

FIGS. 4A and 4B illustrate the different type of movement. FIG. 4A shows a tooth 400 from the front and FIG. 4B of the same tooth 400 from the side.

FIGS. 4A and 4B shows three axes. These are: a rotation axis 402, an inclination axis 404, and an angulation axis 406. Values of tooth movement for rotation, inclination and angulation represent angular movement about these axes respectively. FIG. 4A and 4B also show three different translation directions. A first translation 408 is along the inclination axis in either mesial direction (roughly left on the page in FIG. 4A) or the distal direction (roughly right on the page in FIG. 4A). A second translation 410 is along the rotation axis in either the intrusion direction (roughly up the page in FIG. 4A and B) or in the extrusion direction (roughly down the page in FIG. 4A and B). A third translation 412 is along the angulation axis in either the buccal direction (roughly left on the page in FIG. 4B) or the lingual direction (roughly right on the page in FIG. 4B).

There is a need to encode the type of movement, and the direction of the movement, into a numeric format in order for the data to be compared (e.g. for the planned and actual movements or deviations to be compared). This is shown in table 1 below. For example, inclination in the lingual direction is encoded as a plus and inclination in the buccal direction is encoded as a negative.

TABLE 1

| | | Transformation into |
|---|---|---|
| Variable | Non-numeric code | numeric format |
| Inclination | L (Lingual) | + |
| | B (Buccal) | − |
| Angulation | M (Mesial) | + |
| | D (Distal) | − |
| Rotation | D (Distal) | + |
| | M (Mesial) | − |
| Translation | M (Mesial) | + |
| (Mesial/Distal) | | |
| | D (Distal) | − |
| Translation | B (Buccal) | + |
| (Buccal/Lingual) | | |
| | L (Lingual) | − |
| Translation | E (Extrusion) | + |
| (Intrusion/Extrusion) | | |
| | I (Intrusion) | − |

The use of non-dental orthodontic reference points in the superposition step was described above. The inventors have identified a number of different such reference points which can be used in the method. These will now be described.

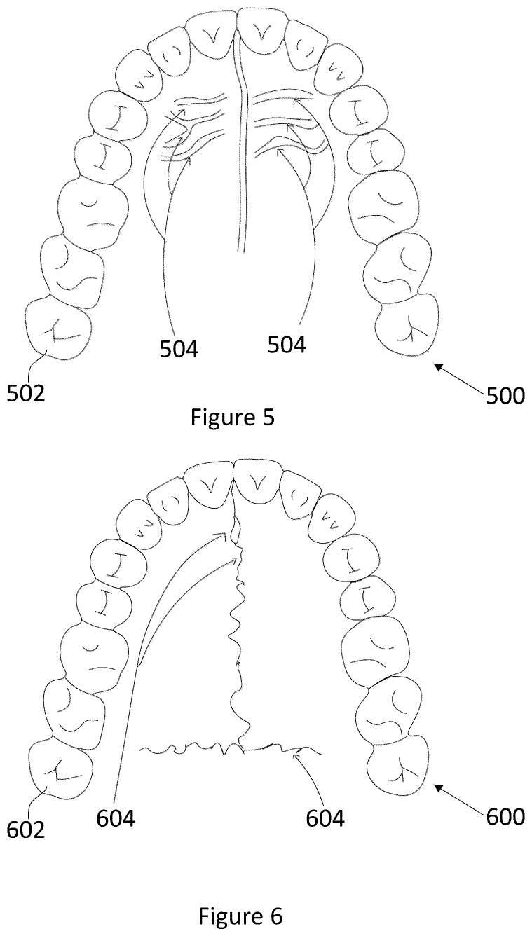

FIG. 5 shows a model of the top jaw 500 of a subject. A plurality of teeth 502 are shown. FIG. 5 further shows soft tissue structures 504 (or soft tissue features) of the subject's palate. These soft tissue feature may be described as anatomical landmarks. One or more structures 504 may be identified as being present in both the first and second models and used as dental orthodontic reference points. These can then be used in an alignment or superposition method.

FIG. 6 shows a model of the top jaw 600 of a subject. A plurality of teeth 602 are shown. FIG. 6 further shows hard tissue structures 604 (or hard tissue features) of the subject's palate. These hard tissue feature may be described as anatomical landmarks. One or more structures 604 may be identified as being present in both the first and second models and used as dental orthodontic reference points. These can then be used in an alignment or superposition method.

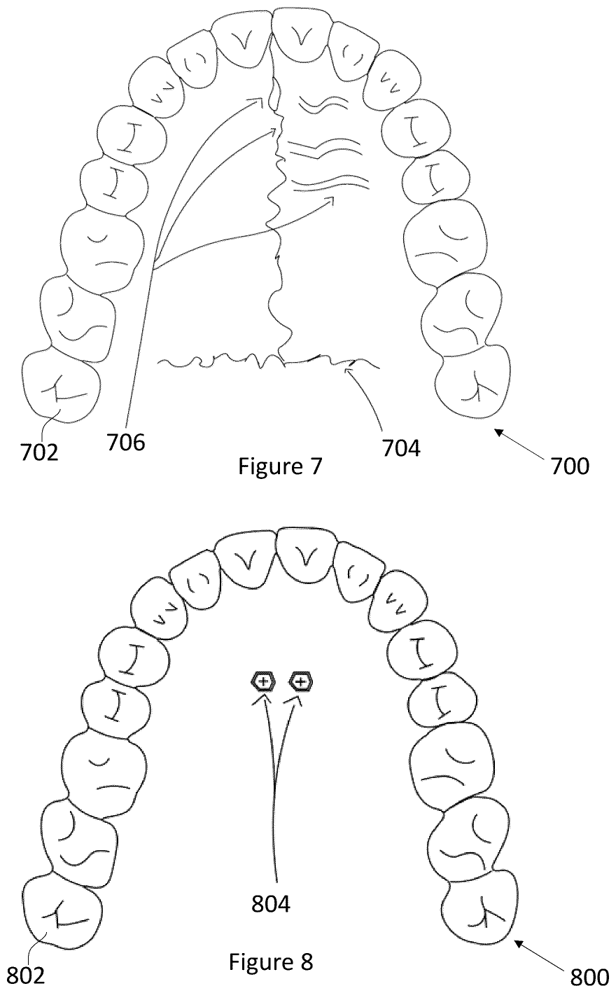

FIG. 7 shows a model of the top jaw 700 of a subject. A plurality of teeth 702 are shown. FIG. 7 further shows hard tissue structures 704 (or hard tissue features) of the subject's palate as well as soft tissue structures 706 (or soft tissue features). FIG. 7 effectively comprises the combination of the soft and hard features of FIGS. 5 and 6. In some examples, building a model comprising both type of landmark/structure may require receiving data from a plurality of types of (synchronized) scans. One or more of the structures 704, 706 may be identified as being present in both the first and second models and used as dental orthodontic reference points. These can then be used in an alignment or superposition method.

FIG. 8 shows a model of the bottom jaw 800 of a subject. A plurality of teeth 802 are shown. FIG. 8 further shows temporary anchorage points or devices 804 fitted to the bottom jaw 800 of the subject. The temporary anchorage points 804 may be being present in both the first and second models and used as dental orthodontic reference points. Thus, in this example, the reference points are artificial rather than anatomical. These reference points can then be used in an alignment or superposition method. While FIG. 8 shows the temporary anchorage points 804 in the middle of the jaw, it should be understood that these could be placed in any convenient and stable position. Another such position could be immediately behind the molars, for example.

The training of a machine learning algorithm 102 has been described above. In a specific example, the machine learning algorithm 102 is a feedforward neural network. In particular, the neural network is a multilayer perceptron (MLP) neural network in this example. The MLP neural network can be constructed using TensorFlow's Keras API. For each outcome variable, a grid search may be performed to optimize parameters (alpha, number of iterations, activation function, learning rate, hidden layer sizes). A Multi-Layer Perceptron (MLP) regressor is chosen as the model, configured with parameters obtained through the previous grid search optimization. KFold cross-validation with 10 folds is chosen to evaluate the model's performance robustly. This technique partitions the dataset into 10 subsets, iteratively training the model on 9 subsets and testing it on the remaining subset.

Mean Squared Error (MSE) and R-squared scores can be calculated for each fold, providing insights into the model's predictive accuracy and goodness of fit. Throughout the cross-validation process, the model with the highest R-squared score can be identified and saved for further analysis. Both the best-performing model and its performance metrics can be saved, facilitating reproducibility and future reference.

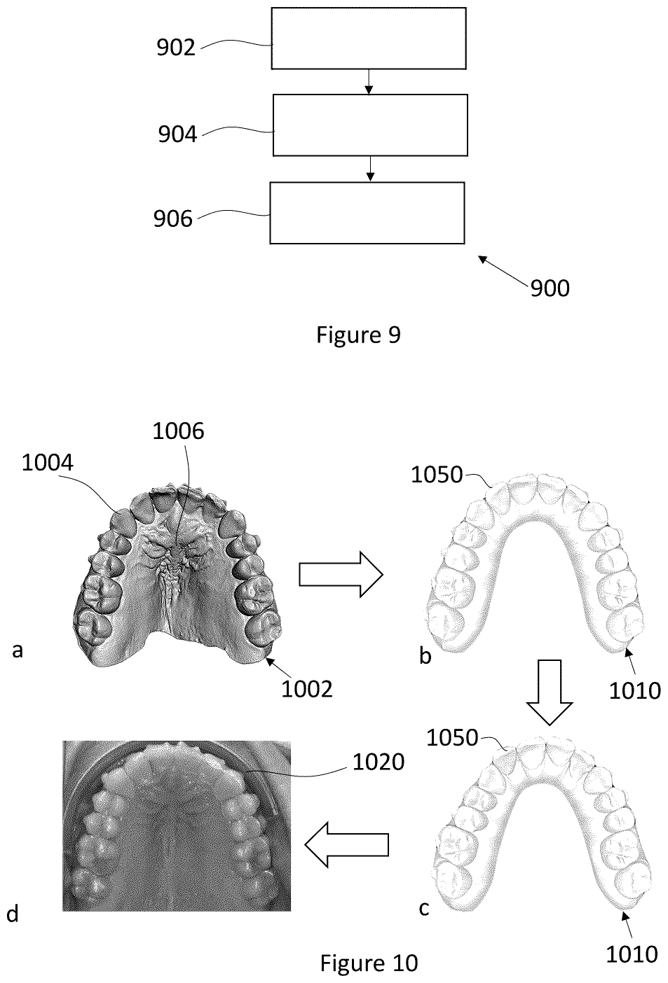

FIG. 9 shows a flow chart 900 of a method of determining improved tooth movements using the machine learning algorithm 102 trained as described above. The tooth movements being “improved” is relative to planned tooth movements determined using conventional software such as ClinCheck Pro® from Invisalign®. In general terms, the method comprises inputting said planned tooth movements into the machine learning algorithm to improve them.

In more detail, step 902 of the method comprises receiving, at a processor, one or more planned tooth movements for teeth of the subject. These are the tooth movements that have been determined previously, e.g. using ClinCheck Pro® from Invisalign®.

Step 904 comprises extracting, using a machine learning model trained in accordance with the method of FIG. 2, one or more predicted deviations based at least in part on the planned tooth movements. The or each predicted deviation represents one or more differences between the planned tooth movements and expected tooth movements if the initial planned tooth movements were used in a tooth alignment process.

Step 906 of the method comprises determining, using a processor, improved planned tooth movements. In examples, this comprises combining (e.g. adding or subtracting) the predicted deviation to the planned tooth movements. For example, a predicted deviation in inclination for tooth may be +10 degrees. This indicates that the model should undercompensate for inclination of this tooth. Thus, the step of improving the planned tooth movements might be to reduce the planned inclination movement of tooth A by 10 degrees to compensate for the predicted deviation. After step 906, the improved tooth movements are used in a real-world aligner treatment.

In some examples, this includes the step of communicating, using a communication device, data associated with the improved tooth movements to a fabrication device for manufacturing an aligner tray. In some examples, this includes step of fabricating, using a fabrication device, one or more aligner trays in accordance with the improved tooth movements. This may comprise 3D printing the one or more aligner trays, for example. In some examples, this includes the step of creating or updating a CAD model for manufacturing an aligner tray using the improved planned tooth movements.

FIGS. 10A to 10D represents some stages of the process of determining improved tooth movements.

FIG. 10A shows a scan of the upper jaw 1002 of a subject, comprising teeth 1004 and palate 1006. FIG. 10B shows a model 1010 of the upper jaw that has been formed using the scan data of FIG. 10A and using planned tooth movements output by aligner software. FIG. 10C shows a model 1020 of the upper jaw using improved tooth movements output by the trained machine learning algorithm described previously. Particularly notable, it can be seen that tooth 1050 is in a different position in FIG. 10B to FIG. 10C. This is to compensate for a deviation determined/predicted by the machine learning algorithm. FIG. 10D is a photograph showing the upper jaw of the subject in which an aligner tray 1020 is in place, the aligner tray is fabricated to achieve the movement represented by FIG. 10C.

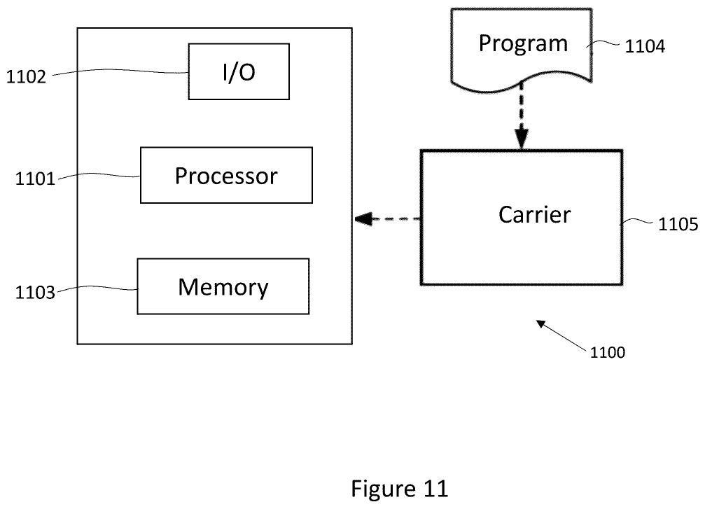

Turning now to FIG. 11, which schematically illustrates a tooth movement improvement unit 1100. The tooth movement improvement unit 1100 comprises an input/output circuitry 1102, at least one processor 1101 and a memory 1103. The memory 1103 contains instructions executable by the processor 1101, cause the tooth movement improvement unit 1100 to perform one or more of the methods of the training and/or the prediction described above.

The instructions that are executable by the processor 1101 may be software in the form of a computer program 1104. The computer program 1104 may be contained in or by a carrier 1105, which may provide the computer program 1101 to the memory 1103 and processor 1101. The carrier 1105 may be in any suitable form including an electronic signal, an optical signal, a radio signal or a computer readable storage medium.

As used herein, the term “computer readable medium” may be a universal serial bus (USB) memory, a digital versatile disc (DVD), a Blu-ray disc, a software module that is received as a stream of data, a Flash memory, a hard drive, a memory card, such as a MemoryStick, a multimedia card (MMC), secure digital (SD) card, etc. One or more of the aforementioned examples of computer readable medium may be provided as one or more computer program products.

1. A method of training a machine learning model to improve tooth movements for a process of tooth alignment of a subject, the method comprising:
receiving, in a computer memory, for a plurality of subjects:
a) one or more planned tooth movements for a tooth alignment process for aligning one or more teeth of the respective subject;
b) first scan data obtained in a first scan of the respective subject before a tooth alignment process using the respective planned tooth movements has been performed; and
c) second scan data obtained in a second scan of said one or more teeth of the respective subject after the tooth alignment process has been performed;
wherein both the first and second scan data comprises scan data for said one or more teeth of the respective subject and scan data for one or more non-dental orthodontic reference points of the respective subject;
wherein the method comprises:
determining, for one or more of the subjects, actual tooth movements of the respective one or more teeth using the first and second scan data and the non-dental orthodontic reference points;
determining deviations between the planned tooth movements and the actual tooth movements; and
training one or more machine learning models at least in part using planned tooth movements of one or more subjects, the one or more machine learning models being arranged to extract one or more predicted deviations based at least in part on the planned tooth movements;
wherein the or each predicted deviation represents one or more predicted differences between the planned tooth movements and expected tooth movements if the planned tooth movements were used in a tooth alignment process.

2. A method as defined in claim 1, wherein the one or more non-dental orthodontic reference points comprise at least one of: an anatomical landmark of the respective subject and a temporary anchorage point received by the respective subject.

3. A method as defined in claim 1, wherein the one or more non-dental orthodontic reference points comprise one or more anatomical landmarks comprising at least one of: a feature or portion of the palate such as soft or hard tissue, a feature or portion of the median suture, a feature or portion of the incisive foramen, one or more anterior contours of the chin, one or more trabecular structures of the mandibular canal and symphysis, one or more inner cortical structures at the inferior border of the symphysis, and one or more lower contours of a molar germ.

4. A method as defined in claim 1, wherein determining the actual tooth movements comprises forming a first model of the respective one or more teeth based on the first data; and forming a second model of the respective one or more teeth based on the second data.

5. A method as defined in claim 4, wherein first and second models comprise the one or more non-dental orthodontic reference points.

6. A method as defined in claim 5, wherein determining the actual tooth movements comprises superimposing or aligning the non-dental orthodontic reference points of the first model and the non-dental orthodontic reference points of the second model.

7. A method as defined in claim 4, wherein determining the actual tooth movements comprises comparing at least one of a position and orientation of a tooth or teeth of first and second models.

8. A method as defined in claim 1, wherein the planned tooth movements and the actual tooth movements comprise movements for a plurality of teeth, optionally at least 10 teeth, optionally at least 20 teeth, optionally all teeth of the respective subject.

9. A method as defined in claim 1, wherein the planned tooth movements have been obtained using a computer-aided design CAD package for modelling virtual tooth movement.

10. A method as defined in claim 1, wherein the or each planned tooth movement and/or the or each actual tooth movement comprises at least one of: a value representing the change in inclination, angulation, rotation, or translation of the respective tooth from an initial position to a final position.

11. A method as defined in claim 1, wherein the machine learning model is a deep neural network DNN.

12. A method of training a machine learning model to determine tooth movements for a process of tooth alignment of a subject, the method comprising:
receiving, in a computer memory, for a plurality of subjects:
a) planned tooth movements for a tooth alignment process for aligning one or more teeth of the respective subject; and
b) actual tooth movements for said one or more teeth of the respective subject after a tooth alignment process has been performed;
the method further comprising training one or more machine learning models at least in part using the planned tooth movements, the one or more machine learning models being arranged to extract one or more predicted deviations based at least in part on the planned tooth movements;
wherein the or each predicted deviations represent differences between the planned tooth movements and expected tooth movements if the planned tooth movements were used in a tooth alignment process.

13. A system for determining improved tooth movements for a process of tooth alignment of a subject, the system comprising one or more processors and a memory comprising instructions which, when executed by the one or more processors, cause the system to carry out the steps of the method of claim 1.

14. A method of determining improved tooth movements for a process of tooth alignment of a subject, the method comprising:
receiving, at a processor, one or more planned tooth movements of one or more teeth of the subject;
extracting, using a machine learning model trained in accordance with the method as defined in claim 1, one or more predicted deviations based at least in part on the planned tooth movements; and
determining, using a processor, improved planned tooth movements;
wherein the or each predicted deviation represents one or more differences between the planned tooth movements and expected tooth movements if the initial planned tooth movements were used in a tooth alignment process; and
wherein the improved planned tooth movements are based at least in part on the planned tooth movements and the predicted deviations.

15. The method of claim 14 further comprising communicating, using a communication device, data associated with the improved tooth movements to a fabrication device for manufacturing an aligner tray.

16. The method of claim 14, further comprising creating or updating a CAD model for manufacturing an aligner tray using the improved planned tooth movements.

17. The method of claim 14, comprising fabricating, using a fabrication device, one or more aligner trays in accordance with the improved tooth movements.

18. A system for determining improved tooth movements for a process of tooth alignment of a subject, the system comprising one or more processors and a memory comprising instructions which, when executed by the one or more processors, cause the system to carry out the steps of the method of claim 14.
